(19) United States
(12) Patent Application Publication
(10) Pub. No.: US 2023/0414543 A1
(43) Pub. Date: Dec. 28, 2023

(54) METHOD OF FEEDING A GESTATING OR LACTATING ANIMAL

(71) Applicant: NUTRECO IP ASSETS B.V., Boxmeer (NL)

(72) Inventors: Pieter LANGENDIJK, AMERSFOORT (NL); John Henry DOELMAN, AMERSFOORT (NL); Néstor Augusto GUTIÉRREZ CÉSPEDES, AMERSFOORT (NL)

(21) Appl. No.: 18/244,811

(22) Filed: Sep. 11, 2023

Related U.S. Application Data
(63) Continuation of application No. PCT/EP22/56356, filed on Mar. 11, 2022.

(30) Foreign Application Priority Data
Mar. 12, 2021 (EP) 21162334.3

(51) Int. Cl. A61K 31/191; A23K 50/30; A23K 20/105

(52) U.S. Cl. CPC A61K 31/191; A23K 50/30; A23K 20/105

(57) ABSTRACT

The present invention pertains to the field of maximizing performance of animals, particularly of gestating or lactating animals and their offspring, particularly of gestating or lactating sows and their offspring. Particularly the present invention is in the field of increasing colostrum production, milk production, colostral- and/or milk protein yield, colostral and/or milk total solids yield, said total solids being the total of fat, protein and lactose, in colostrum and/or milk, colostral- and/or milk lactose yield, and average daily gain of the offspring, improving survival of the offspring and increasing number of offspring at weaning.

### FIELD OF THE INVENTION

The present invention pertains to the field of maximizing performance of animals, particularly of gestating or lactating animals and their offspring, particularly of gestating or lactating sows and their offspring. Particularly the present invention is in the field of increasing colostrum production, milk production, colostral- and/or milk protein yield, colostral and/or milk total solids yield, said total solids being the total of fat, protein and lactose, in colostrum and/or milk, colostral- and/or milk lactose yield, and average daily gain of the offspring, improving survival of the offspring and increasing numbers of offspring at weaning.

### BACKGROUND OF THE INVENTION

Maximizing the performance of gestating or lactating monogastric animals and their offspring has been a major objective of nutritionists for a long time. Important aspects of maximizing reproductive performance is to optimize colostrum production in a gestating monogastric animal and/or milk production in a lactating monogastric animal. Colostrum is produced by the mammary glands of the animal and is secreted between parturition and 24 hours thereafter. After the colostrum has been secreted, the mammary glands will produce milk for the offspring. The animal then has reached the lactation phase.

It is known to the person skilled in the art that colostrum has a different and antibody-rich composition, compared to milk produced through the lactation phase, and both have a crucial impact on the performance of the offspring. It is believed that the amount of colostrum consumed by the offspring at the very beginning of life has a positive impact on its performance during the rest of life.

It is an object of the present invention to provide a method of feeding a composition to a gestating or lactating monogastric animal, preferably a gestating or lactating mammal, preferably a gestating or lactating sow, a method to produce such composition, and use of the composition for increasing colostrum production, milk production, colostral- and/or milk protein yield, colostral and/or milk total solids yield, said total solids being the total of fat, protein and lactose, colostral- and/or milk lactose yield, and average daily gain of the offspring, improving survival of the offspring and increasing numbers of offspring at weaning.

### SUMMARY OF THE INVENTION

The present inventors surprisingly found that use of a composition comprising gluconic acid or an ester or salt thereof, in a diet for a gestating or lactating monogastric animal, preferably a gestating or lactating mammal, preferably a gestating or lactating sow, increased colostrum production, milk production, colostral- and/or milk protein yield, colostral and/or milk total solids yield, said total solids being the total of fat, protein and lactose, colostral- and/or milk lactose yield, and average daily gain of the offspring, improved survival of the offspring and increased numbers of offspring at weaning.

In a first aspect, the present invention relates to a method of feeding a gestating or lactating monogastric animal, comprising administering to the animal a composition comprising gluconic acid or an ester or salt thereof, wherein the amount of gluconic acid or an ester or salt thereof in the composition is intended to provide a dosage in a range of 0.02-100 grams per day.

In an embodiment, the gluconic acid or an ester or salt thereof is administered in the form of a top dress formulation, an animal feed, a premix or supplement or animal drinking water.

In an embodiment, the gluconic acid or an ester or salt thereof is administered to a gestating monogastric animal.

In an embodiment, the gluconic acid or an ester or salt thereof is administered to the gestating monogastric animal in a period from about 45 days prior to parturition until parturition.

In an embodiment, the gluconic acid or an ester or salt thereof is administered to a lactating monogastric animal.

In an embodiment, the gluconic acid or an ester or salt thereof is administered to the lactating monogastric animal in a period from parturition to weaning.

In an embodiment, the gestating or lactating monogastric animal is a gestating or lactating monogastric mammal.

In an embodiment, the monogastric mammal is a sow.

In a further aspect, the present invention relates to a method of producing a composition for feeding a gestating or lactating monogastric animal, comprising mixing gluconic acid or an ester or salt thereof with one or more feed components or one or more feed additives or water, to produce the composition, wherein the amount of gluconic acid or an ester or salt thereof in the composition is intended to provide, or provides, a dosage in a range of 0.02-100 grams per day.

In an embodiment, the composition is selected from a group consisting of a top dress formulation, an animal feed, a premix or supplement and an animal drinking water.

In an embodiment, the gestating or lactating monogastric animal is a gestating or lactating monogastric mammal.

In an embodiment, the monogastric mammal is a sow.

In a further aspect, the present invention relates to the use of gluconic acid or an ester or salt thereof, or of a composition comprising gluconic acid or an ester or salt thereof, in a diet for a gestating or lactating monogastric animal.

In a further aspect, the present invention relates to use of gluconic acid or an ester or salt thereof, or of a composition comprising gluconic acid or an ester or salt thereof, for increasing colostrum production of a monogastric animal.

In a further aspect, the present invention relates to use of gluconic acid or an ester or salt thereof, or of a composition comprising gluconic acid or an ester or salt thereof, for increasing milk production of a monogastric animal.

In a further aspect, the present invention relates to use of gluconic acid or an ester or salt thereof, or of a composition comprising gluconic acid or an ester or salt thereof, for increasing colostral and/or milk protein yield in a monogastric animal.

In a further aspect, the present invention relates to use of gluconic acid or an ester or salt thereof, or of a composition comprising gluconic acid or an ester or salt thereof, for increasing total solids yield, total solids being the total of fat, protein and lactose, in colostrum and/or milk in a monogastric animal.

In a further aspect, the present invention relates to use of gluconic acid or an ester or salt thereof, or of a composition comprising gluconic acid or an ester or salt thereof, for increasing colostral and/or milk lactose yield in a monogastric animal.

In a further aspect, the present invention relates to use of gluconic acid or an ester or salt thereof, or of a composition comprising gluconic acid or an ester or salt thereof, for increasing immunoglubulins yield in colostrum and/or milk in a monogastric animal.

In a further aspect, the present invention relates to use of gluconic acid or an ester or salt thereof, or of a composition comprising gluconic acid or an ester or salt thereof, for increasing average daily gain of offspring of a monogastric animal.

In a further aspect, the present invention relates to a gluconic acid or an ester or salt thereof, or a composition comprising gluconic acid or an ester or salt thereof, for use in improving survival of offspring of a monogastric animal.

In a further aspect, the present invention relates to a gluconic acid or an ester or salt thereof, or a composition comprising gluconic acid or an ester or salt thereof, for use in increasing numbers of offspring at weaning of a monogastric animal.

In an embodiment, the composition is selected from a group consisting of a top dress formulation, an animal feed, a premix, a supplement, and an animal drinking water.

### DETAILED DESCRIPTION OF THE INVENTION

### General Definitions

In the following description and examples, a number of terms are used. In order to provide a clear and consistent understanding of the specification and claims, including the scope to be given to such terms, the following definitions are provided. Unless otherwise defined herein, all technical and scientific terms used have the same meaning as commonly understood by someone skilled in the art to which this invention belongs. The disclosures of all publications, patent applications, patents and other references cited herein are incorporated herein in their entirety by reference.

The term ‘pregnant’ or ‘pregnancy’, also known as ‘gestation’, as used herein refers to the period of time between conception and parturition, wherein the embryo or fetus is developing in the uterus. The duration of the gestation strongly varies from species to species. Smaller animals usually have shorter periods of gestation than larger animals. In pigs the gestation normally lasts for about 115 days, but there can be some variability from case to case. Gestation can be divided into different phases like fertilization at the very beginning, placental formation, fetal development and major fetal growth at the end, before parturition. Another important development during gestation is mammary gland development, which in pigs typically takes place between around day 80 (around 35 days before parturition) and parturition. During that period of time the mammary gland will develop and get ready for the energy demanding lactation phase.

The term ‘monogastric’ as used herein refers to the single-chambered stomach system of monogastric animals. Examples of monogastric animals are animals such as swine or poultry. Contrarily, ruminants have a more complex stomach system, comprising more stomach compartments, such as the rumen, reticulum, omasum and abomasum. Because of the completely different digestive system of monogastrics, the skilled person knows that the monogastric system and its function cannot be compared to the more complex ruminant digestive system. Certain types of feed suitable for monogastrics may not be utilized in ruminants because the fermenting function of the rumen may alter the nutrient profile of the feed prior to absorption. Certain types of feed particularly suitable for ruminants may not be tolerated by monogastrics because such feed may require microbial fermentation to liberate nutrients that are otherwise unavailable to the animal.

The term ‘parturition’, also known as ‘farrowing’, as used herein refers to the process of giving birth. In pigs this process typically ranges from three to eight hours and piglets are usually delivered every 10 to 20 minutes but there is a wide variation. Between the first and second piglet there can be a gap of up to an hour. The process of parturition is activated by the piglets when they reach the final stage of maturity and signal the uterus to produce prostaglandins which are circulated to the ovary and cause luteolysis. This finally results in termination of the pregnancy and activation of parturition.

The term ‘lactation’ as used herein refers to the production of milk by the mammary glands of the lactating animal during a period of time after parturition, for feeding the offspring. In pigs, the lactation period lies between parturition and the moment that piglets are weaned. Typically, piglets are weaned within about 3, 4 or 5 weeks after birth. The moment of the start of weaning may vary between husbandry systems.

The term ‘survival’ as used herein refers to the survival of newborn animals after birth. The term survival comprises ‘neonatal survival’ and ‘survival of offspring through the lactation period’, also known as ‘pre-wean survival’. The term ‘neonatal survival’, as used herein refers to the survival of newborn animals within ten days after birth. It is the opposite of neonatal mortality or neonatal death, which is the phenomenon where offspring dies shortly after birth, typically within ten days after birth. The term ‘pre-wean survival’, as used herein refers to the survival of newborn animals until weaning, which is within about 3, 4 or 5 weeks after birth. It is the opposite of pre-wean mortality or pre-wean death or mortality before weaning, which is the phenomenon where offspring dies after birth, typically within 3, 4 or 5 weeks after birth. Pre-wean survival means that the animal survives the first 3 weeks after birth, preferably the animal survives the first 4 weeks after birth.

The term ‘numbers of offspring at weaning’, as used herein refers to the numbers of offspring that survive the lactation period, which is the period of time between parturition and weaning. If mortality of offspring during lactation is reduced, the survivability will be increased. As a consequence an increased number of offspring will reach weaning.

The term ‘colostrum’, as used herein, refers to the fluid first secreted from the mammary glands between parturition and 24 hours thereafter. It is known to the person skilled in the art that colostrum has a different and antibody-rich composition, compared to milk produced through the lactation phase. Colostrum uptake by the offspring, which typically corresponds to the colostrum production of the pregnant animals, can be calculated based on the increase in body weight of the offspring between birth and 24 hours thereafter, based on the algorithm developed by Theil et al. (Mechanistic model to predict colostrum intake based on deuterium oxide dilution technique data and impact of gestation and prefarrowing diets on piglet intake and sow yield of colostrum. J. Anim. Sci. 2014, 92:5507-5519).

The term ‘milk production’ as used herein, refers to the amount by weight of milk produced by the lactating animal. In animals such as sows, litter weight gain is a well-known measure for milk production. As a measure of milk production, the weight gain of the litter may be measured a few times during lactation, such as for example 24 hours after parturition, 7 days after parturition, and again at 24 days of lactation, just before piglets are weaned, in order to determine the litter weight gain during lactation, and thus the milk production of the sow.

The term ‘colostral protein yield’, as used herein refers to the amount by weight of colostral protein that is produced by the lactating animal in the colostrum between parturition and 24 hours thereafter. It is typically quantified in terms of g/day.

The term ‘milk protein yield’, as used herein refers to the amount by weight of milk protein that is produced by the lactating animal during lactation. It is typically quantified in terms of g/day.

The term ‘total solids yield’ as used herein refers to the total amount of fat, protein and lactose, in colostrum and/or milk in a monogastric animal.

The term ‘colostral fat yield’ as used herein refers to the amount by weight of colostral fat that is produced by the lactating animal in the colostrum between parturition and 24 hours thereafter. It is typically quantified in terms of g/day.

The term ‘milk fat yield’ as used herein refers to the amount by weight of milk fat that is produced by the lactating animal during lactation. It is typically quantified in terms of g/day.

The term ‘colostral lactose yield’ as used herein refers to the amount by weight of colostral lactose that is produced by the lactating animal in the colostrum between parturition and 24 hours thereafter. It is typically quantified in terms of g/day.

The term ‘milk lactose yield’ as used herein refers to the amount by weight of milk lactose that is produced by the lactating animal during lactation. It is typically quantified in terms of g/day.

The term ‘average daily gain’ as used herein refers to a value that shows the average weight gain of an animal per day. It is obtained by dividing how much an animal has grown (weight increase) by the number of days needed to accomplish this growth.

The term ‘gluconic acid’ as used herein refers to an organic compound with molecular formula C₆H₁₂O₇ and condensed structural formula HOCH₂(CHOH)₄COOH. It is one of the 16 stereoisomers of 2,3,4,5,6-pentahydroxyhexanoic acid. The term ‘gluconic acid derivative(s)’ as used herein refers to compound(s) derived from gluconic acid and includes gluconate salts and gluconate esters. The term ‘gluconate salts’ as used herein refers to any salts derived from gluconic acid. The salts of gluconic acids are also known as “gluconates”. Non-limiting examples of gluconate salts include calcium gluconate, sodium gluconate, ferrous gluconate, potassium gluconate, zinc gluconate, copper gluconate, cobalt gluconate, barium gluconate, lithium gluconate, magnesium gluconate, manganese gluconate, cupric gluconate and the like. Non-limiting examples of gluconate esters include gluconic acid cyclic ester with boric acid, quinine gluconate, glucono-delta-lactone, and the like.

The term ‘diet’ as used herein refers to the habitual nourishment of the animal, including feed (solid and liquid feed) and drinking water.

The term ‘supplement’, also known as ‘additive’, as used herein refers to a product intended for ingestion, which contains one or more ingredients intended to add nutritional value to the diet. The supplement may be added to a feed composition, and includes, without limitation, animal feeds, top dresses or premixes. It may also be added to the drinking water (in which case the supplement is often referred to as a drinking water additive).

The term lop dress' as used herein refers to an animal feed which is added on top of the normal diet of the animal. A top dress can be used as a way to add one or more supplements to the diet. Next to the particular one or more supplements, a top dress typically contains materials like barley, wheat, corn, wheat bran, molasses, vegetable oil, and/or sugar.

The term ‘premix’ as used herein refers to a complex mixture of compounds like vitamins, minerals, trace elements, supplements and/or other nutritional additives, for incorporation into feed. The premix is typically incorporated at a level of between 0.2 and 2% (micro premix) or between 2 to 8% (macro premix) into the feed. It is usually mixed with feed in an early stage in the manufacturing and distribution process.

The terms ‘to improve’ or ‘improving’ as used herein refer to the ability to bring in a more desirable state or condition. Someone or something might for example become better or might get better properties or quality. The ability to make things better is also covered in a sense of the ability to ameliorate, like improving a bad situation or quality, or repairing bad or not working properties.

The terms ‘to increase’ and ‘increased level’ and the terms ‘to decrease’ and ‘decreased level’ refer to the ability to increase or decrease a particular amount or number. A level in a test sample may be increased or decreased when it is at least 5%, such as 10%, 15%, 20%, 25%, 30%, 35%, 40%, 45%, 50% higher or lower, respectively, than the corresponding level in a control sample or reference sample.

The term ‘about’, as used herein indicates a range of normal tolerance in the art, for example within 2 standard deviations of the mean. The term “about” can be understood as encompassing values that deviate at most 10%, 9%, 8%, 7%, 6%, 5%, 4%, 3%, 2%, 1%, 0.5%, 0.1%, 0.05%, or 0.01% of the indicated value.

The terms “comprising” or “to comprise” and their conjugations, as used herein, refer to a situation wherein said terms are used in their non-limiting sense to mean that items following the word are included, but items not specifically mentioned are not excluded. It also encompasses the more limiting verb “to consist essentially of” and “to consist of”. Reference to an element by the indefinite article “a” or “an” does not exclude the possibility that more than one of the elements is present, unless the context clearly requires that there be one and only one of the elements. The indefinite article “a” or “an” thus usually means “at least one”.

### Compositions

The present inventors surprisingly found that use of gluconic acid or an ester or salt thereof, or of a composition comprising gluconic acid or an ester or salt thereof, in a diet for a gestating or lactating monogastric animal, preferably a gestating or lactating mammal, preferably a gestating or lactating sow, increased colostrum production, milk production, colostral- and/or milk protein yield, total solids yield, said total solids being the total of fat, protein and lactose, in colostrum and/or milk, colostral- and/or milk lactose yield, and average daily gain of the offspring, improved survival of the offspring and increased numbers of offspring at weaning.

In a first aspect, the present invention relates to a method of feeding a gestating or lactating monogastric animal, comprising administering to said animal a gluconic acid or an ester or salt thereof, or of composition comprising gluconic acid or an ester or salt thereof, wherein the amount of gluconic acid or an ester or salt thereof in the composition is intended to provide a dosage in a range of 0.02-100 grams per day. Preferably, the dosage of gluconic acid or an ester or salt thereof is in a range of 0.1-50 grams per day, more preferably in a range of 0.7-20 grams per day, more preferably in a range of 1-10 grams per day, more preferably in a range of 1.5-8 grams per day, even more preferably in a range of 2-6 grams per day.

The monogastric animal is preferably a monogastric mammal, even more preferably a sow. It is not relevant in which form the gluconic acid or an ester or salt thereof is added to the diet as long as the animal receives the necessary daily dose of gluconic acid or an ester or salt thereof. Pregnant sows for example eat about 2.5 to 3.5 kg feed a day and drink about 7 litres of drinking water a day. This means that the amount of gluconic acid or an ester or salt thereof to be added either to the feed, or to the drinking water, or to both the feed and drinking water, can easily be determined in order to result in the necessary daily dose of the invention.

In an embodiment, the gluconic acid or an ester or salt thereof is added to an animal feed, wherein the dosage is in a range of 0.02-100 grams per kilogram animal feed. Preferably, the dosage of gluconic acid or an ester or salt thereof is in a range of 0.1-70 grams per kg animal feed, more preferably in a range of 0.3-50 grams per kg animal feed, more preferably in a range of 0.5-10 grams per kg animal feed, more preferably in a range of 0.7-2.5 grams per kg animal feed, even more preferably in a range of 0.9-1.7 grams per kg animal feed.

In an embodiment, the gluconic acid or an ester or salt thereof is a gluconic acid salt, selected from calcium gluconate, sodium gluconate, ferrous gluconate, potassium gluconate, zinc gluconate, copper gluconate, cobalt gluconate, barium gluconate, lithium gluconate, magnesium gluconate, manganese gluconate and/or cupric gluconate, preferably the gluconic acid salt is selected from calcium gluconate or sodium gluconate.

In an embodiment, the gluconic acid or an ester or salt thereof is a gluconic acid ester selected from gluconic acid cyclic ester with boric acid, quinine gluconate and/or glucono-delta-lactone.

It is known to the person skilled in the art that animal feed can be formulated in many different ways, which means supplements can be added to the feed in different ways.

In an embodiment, gluconic acid or an ester or salt thereof is added to an animal feed as a top dress formulation. Top dresses are typically added to the feed in a certain amount per kilogram feed. A non-limiting example is the addition of 100 grams of top dress to 1 kg of feed. In that case, for a dosage of 0.2 gram gluconic acid per kg of feed, 100 grams of top dress should contain 0.2 grams of gluconic acid in order to formulate the necessary dose per kilogram feed. When in another non-limiting example feed is formulated containing 0.4 grams of gluconic acid per kilogram feed, by the use of 50 grams of a top dress formulation per kilogram of feed, the top dress formulation should contain 0.4 grams of gluconic acid in 50 grams of top dress. The top dress may comprise one or more additional components in addition to the composition as taught herein. These one or more additional components may serve as a carrier material for the composition as taught herein. Non-limiting examples of the one or more additional components are barley, wheat, corn, wheat bran, molasses, vegetable oil, sugar, chalk and the like. In an embodiment, the top dress comprises the composition as taught herein without one or more additional components. In an embodiment the top dress comprises the composition as taught herein, wherein the dosage of the composition is 0.1-100%, based on the total weight of the said top dress, preferably the dosage of the composition is 0.2-75%, based on the total weight of the said top dress, more preferably the dosage of the composition is 0.3-50%, based on the total weight of the said top dress, more preferably the dosage of the composition is 0.4-30%, based on the total weight of the said top dress, even more preferably the dosage of the composition is 0.5-20%, based on the total weight of the said top dress, most preferably the dosage of the composition is 0.6-10%, based on the total weight of the said top dress.

Alternatively, gluconic acid or an ester or salt thereof may be added to an animal feed by direct addition of the composition in the feed formulation process, or by the use of a supplement or premix. A premix, which is a complex mixture of compounds, may be incorporated in feed in certain percentages during the feed formulation process. The percentages of incorporation can vary from one feed to the other. A non-limiting example is a feed comprising 1 gram gluconic acid per kg feed, formulated by addition of 1% of premix into the animal feed. In case of 1% incorporation, the premix should contain 100 grams of gluconic acid for 1 kilogram of premix, in order to end up with a dosage of 1 gram of gluconic acid per kilogram feed. The supplement or premix may comprise one or more additional components in addition to the composition as taught herein. These one or more additional components may serve as a carrier material for the composition as taught herein. Non-limiting example of the one or more additional components are vitamins, minerals, trace elements, supplements, other nutritional additives, chalk and the like. In an embodiment, the supplement or premix comprises the composition as taught herein without one or more additional components. In an embodiment the supplement or premix comprises the composition as taught herein, wherein the dosage of the composition is 5-100%, based on the total weight of the said supplement or premix, preferably the dosage of the composition is 3-90%, based on the total weight of the said supplement or premix, more preferably the dosage of the composition is 6-80%, based on the total weight of the said supplement or premix, more preferably the dosage of the composition is 9-70%, based on the total weight of the said supplement or premix, even more preferably the dosage of the composition is 15-60%, based on the total weight of the said supplement or premix, most preferably the dosage of the composition is 20-45%, based on the total weight of the said supplement or premix. In an embodiment, the gluconic acid or an ester or salt thereof is added to an animal drinking water wherein the dosage is in a range of 0.01-100 grams per liter of animal drinking water. Preferably, the dosage of gluconic acid or an ester or salt thereof is in a range of 0.01-50 grams per liter animal drinking water, more preferably in a range of 0.02-20 grams per liter animal drinking water, more preferably in a range of 0.02-8 grams per liter animal drinking water, more preferably in a range of 0.05-3 grams per liter animal drinking water, even more preferably in a range of 0.05-1.5 grams per liter animal drinking water.

In an embodiment, the composition as taught herein may be added to the diet of animals by adding part of the daily dose to the animal feed and the other part of the daily dose to the drinking water.

In an embodiment, the gluconic acid or an ester or salt thereof may be presented in the form of a core and may be coated with an agent, or the gluconic acid or an ester or salt thereof may be embedded in a matrix of an agent.

In an embodiment, the gluconic acid or an ester or salt thereof is prepared by embedding the gluconic acid or an ester or salt thereof in a matrix of an agent, e.g., a vegetable oil, e.g., an at least partially hydrogenated vegetable oil, e.g., a hydrogenated vegetable oil. The vegetable oil may be any vegetable oil, but is preferably selected from the group consisting of palm oil, soybean oil, rape seed oil, cottonseed oil, and castor oil, or mixtures thereof. In a preferred embodiment, preferably said agent comprises or consists of palm oil. In a preferred embodiment, the vegetable oil is partly hydrogenated, preferably the vegetable is fully hydrogenated.

Embedding a gluconic acid or an ester or salt thereof in a matrix of an agent may be done by any technique suitable for making particles from a few microns to several millimetres known to a person skilled in the art. A non-limiting but highly suitable exemplary technique is spray chilling, also referred to as spray cooling, spray congealing, or prilling. Spray chilling is a lipid based system where the active ingredient (e.g., gluconic acid or an ester or salt thereof) is mixed into a molten matrix (e.g., a molten matrix of the agent, such as hydrogenated vegetable oil), which mixture is subsequently fed through a nozzle, e.g., an atomizer nozzle, to produce droplets of the mixture. The droplets are allowed to solidify, e.g., by contacting them with cooled air at a temperature below the melting point of the agent resulting in the formation of particles. In an embodiment, the composition taught herein is obtainable by such method.

When preparing the composition taught herein, it may be advantageous, although not essential, for further facilitating stability of the matrix or coating composition to add one or more ingredients to the said matrix or coating composition. Representative, non-limiting examples of such ingredients include lecithin, waxes (e.g. carnauba wax, beeswax, natural waxes, synthetic waxes, paraffin waxes, and the like), fatty acid esters, magnesium carbonate, calcium carbonate, calcium phosphate, calcium pyrophosphate, calcium hydrogen phosphate hydrates, calcium hydrogen phosphate dihydrate, calcium dihydrogen pyrophosphate, magnesium pyrophosphate, magnesium hydrogen phosphate hydrates, aluminium phosphate, magnesium hydroxide, aluminium hydroxide, manganese oxide, zinc oxide, sodium hydrogen carbonate, and ferric oxide, and mixtures thereof, and others. The skilled person knows how to select suitable ingredients to achieve this purpose.

In an embodiment, the composition taught herein has an average particle size distribution of between about 150 and 3000 μm, such as between about 300 and 2000 μm, or between about 500 and 1500 μm, preferably between 650 and 1250 μm, more preferably between about 800 and 1000 μm. The particle size distribution can be measured by using standard sieve analysis (e.g., using a Retsch Sieve Shaker AS 200), e.g. as taught in ASTM C136. Reference herein to the average particle size is to the average particle diameter.

In an embodiment, the weight percent ratio of the gluconic acid or an ester or salt thereof to the agent ranges from about 20:80 to about 65:35 percent by weight, or is at least about 40:60 percent by weight, preferably about 50:50 percent by weight of the composition taught herein.

In an aspect, the invention relates to a method of producing a composition for feeding a gestating or lactating monogastric animal, comprising mixing gluconic acid or an ester or salt thereof with one or more feed components or one or more feed additives or water, to produce the composition, wherein the amount of gluconic acid or an ester or salt thereof in the composition is intended to provide a dosage in a range of 0.02-100 grams per day.

In an embodiment, the gluconic acid or an ester or salt thereof may be presented in the form of a core and may be coated with an agent, or the gluconic acid or an ester or salt thereof may be embedded in a matrix of an agent.

In an embodiment, the composition is selected from a group consisting of a top dress formulation, an animal feed, a premix or supplement and an animal drinking water.

In an embodiment, the gestating or lactating monogastric animal is a gestating or lactating monogastric mammal. In an embodiment, the monogastric mammal is a sow.

### Methods of Using the Composition

In an aspect, the invention relates to a method of feeding a gestating or lactating monogastric animal, comprising administering to the animal gluconic acid or an ester or salt thereof or a composition as taught herein.

In an embodiment, said gluconic acid or an ester or salt thereof, or the composition as taught herein, is administered to a gestating monogastric animal.

In an embodiment, said gluconic acid or an ester or salt thereof, or the composition as taught herein, is administered to the gestating monogastric animal in a period from about 45 days prior to parturition until parturition. In an embodiment, said gluconic acid or an ester or salt thereof, or the composition as taught herein, is administered to the gestating monogastric animal in a period from about 40 days prior to parturition until parturition. In an embodiment, said gluconic acid or an ester or salt thereof, or the composition as taught herein, is administered to the gestating monogastric animal in a period from about 35 days prior to parturition until parturition.

In an embodiment, said gluconic acid or an ester or salt thereof, or the composition as taught herein, is administered to the lactating monogastric animal.

In an embodiment, said gluconic acid or an ester or salt thereof, or the composition as taught herein, is administered to the lactating monogastric animal in a period from parturition to weaning. In an embodiment, said gluconic acid or an ester or salt thereof, or the composition as taught herein, is administered to the lactating animal in a period from 24 hours after parturition to weaning. In an embodiment, said gluconic acid or an ester or salt thereof, or the composition as taught herein, is administered to the lactating animal in a period from two days after parturition to weaning.

In an aspect, the invention relates to the use of said gluconic acid or an ester or salt thereof, or a composition as taught herein, in a diet for a gestating or lactating monogastric animal, preferably a monogastric mammal, preferably a sow. The inventor surprisingly found that said gluconic acid or an ester or salt thereof, or a composition as taught herein, can be used for non-medical as well as medical purposes.

In another aspect, the invention relates to use of said gluconic acid or an ester or salt thereof, or a composition as taught herein, for increasing colostrum production of a monogastric animal. It is known to the person skilled in the art that the offspring consumes more colostrum if more colostrum is available to the newborn animals. A higher colostrum uptake (also referred to as “intake”, i.e., the amount ingested) by the litter therefore means that more colostrum was produced by the pregnant animal.

In another aspect, the invention relates to use of said gluconic acid or an ester or salt thereof, or a composition as taught herein, for increasing milk production of a monogastric animal.

In another aspect, the invention relates to use of said gluconic acid or an ester or salt thereof, or a composition as taught herein, for increasing colostral and/or milk protein yield in a monogastric animal. In an embodiment, the invention relates to use of said gluconic acid or an ester or salt thereof, or a composition as taught herein, for increasing colostral protein yield in a monogastric animal. In an embodiment, the invention relates to use of said gluconic acid or an ester or salt thereof, or a composition as taught herein, for increasing milk protein yield in a monogastric animal.

In another aspect, the invention relates to use of said gluconic acid or an ester or salt thereof, or a composition as taught herein, for increasing total solids yield, said total solids being the total of fat, protein and lactose, in colostrum and/or milk in a monogastric animal. In an embodiment, the invention relates to use of a composition as taught herein, for increasing total solids yield, said total solids being the total of fat, protein and lactose, in colostrumin a monogastric animal. In an embodiment, the invention relates to use of said gluconic acid or an ester or salt thereof, or a composition as taught herein, for increasing total solids yield, said total solids being the total of fat, protein and lactose, in milk in a monogastric animal.

In another aspect, the invention relates to use of said gluconic acid or an ester or salt thereof, or a composition as taught herein, for increasing colostral and/or milk lactose yield in a monogastric animal. In an embodiment, the invention relates to use of said gluconic acid or an ester or salt thereof, or a composition as taught herein, for increasing colostral lactose yield in a monogastric animal. In an embodiment, the invention relates to use of said gluconic acid or an ester or salt thereof, or a composition as taught herein, for increasing milk lactose yield in a monogastric animal.

In another aspect, the invention relates to use of said gluconic acid or an ester or salt thereof, or a composition as taught herein, for increasing average daily gain of offspring of a monogastric animal.

In another aspect, the invention relates to gluconic acid or an ester or salt thereof, or a composition as taught herein, for use in improving survival of offspring of a monogastric animal. In an embodiment, the invention relates to gluconic acid or an ester or salt thereof, or a composition as taught herein, for use in improving neonatal survival of offspring of a monogastric animal. In an embodiment, the invention relates to gluconic acid or an ester or salt thereof, or a composition as taught herein, for use in improving pre-wean survival of offspring of a monogastric animal.

In another aspect, the invention relates to gluconic acid or an ester or salt thereof, or a composition as taught herein, for use in increasing numbers of offspring at weaning of a monogastric animal.

The present invention is further illustrated, but not limited, by the following examples. From the above discussion and the examples, one skilled in the art can ascertain the essential characteristics of the present invention, and without departing from the teaching and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### EXAMPLES

### Example 1

From day 80 of gestation up to parturition (day 115 of gestation), and subsequently through the whole period of lactation from parturition until weaning (25 days after parturition), sows of various parities were fed a top dress of 100 g/day on top of their normal diet. Sows were allocated to either the placebo group (receiving a placebo top dress), or the treatment group (receiving a top dress comprising calcium gluconate), making sure that sows with different parities were distributed evenly across the two groups. The placebo top dress was formulated from barley, wheat, corn, wheat bran, molasses, vegetable oil, and sugar. In the treatment group, 10% (10 g per 100 g) of the placebo top dress was replaced with the ingredient of interest, calcium gluconate, embedded in a fat matrix. The ingredient (40%) was embedded in a matrix of hydrogenated fat (50%) and calcium carbonate (10%).

TABLE 1

| | Daily dose of |
|---|---|
| | calcium gluconate |
| Ingredient | in 100 g top dress |
| placebo (n = 49) | 0 g |
| calcium gluconate (n = 41) | 4 g |

During gestation, sows were fed the normal diet at approximately 3 kg/day. In lactation, all sows were fed the normal diet at an increasing level following a set feeding scheme, starting from 3 kg after parturition, and increasing by 0.5 kg each day to the maximum intake capacity of each sow. The allowance was capped at a maximum of 9 kg/day.

At parturition, piglets were weighed immediately as they were born, and again at 24 h after birth, to estimate colostrum intake based on the algorithm developed by Theil et al. (Mechanistic model to predict colostrum intake based on deuterium oxide dilution technique data and impact of gestation and prefarrowing diets on piglet intake and sow yield of colostrum. J. Anim. Sci. 2014, 92:5507-5519). One day after parturition, when 24-h weights had been recorded, litter size was standardized by cross-fostering, to 14 or 15 piglets per litter. Piglets were only cross-fostered to sows within the same treatment (either placebo or calcium gluconate treatment). As a measure of milk production, the weight gain of the litter was measured at 7 days after parturition, and again at 24 days of lactation, just before piglets were weaned. Piglets did not receive any additional feed such as creep feed or milk replacer during lactation. When analyzing the data, litter weight gain was corrected for litter size.

Sows gave birth to 14.6 live born piglets on average. During the first seven days of lactation, the litter weight gain was increased by 7.6% for sows that received calcium gluconate compared to those that received the placebo treatment. Similarly, the litter weight gain over the whole lactation period was greater in sows receiving calcium gluconate, and was 5% greater than for placebo sows (Table 2). Neonatal survival, and survival throughout the whole lactation period was greater in litters from sows that were supplemented with calcium gluconate. As a result, litter size at weaning was 0.3 greater in sows supplemented with calcium gluconate compared to those receiving the placebo treatment.

TABLE 2

| | | | | Litter | | | Litter |
|---|---|---|---|---|---|---|---|
| | | Total | | weight | | | weight |
| | | colostrum | | gain | | | gain |
| | Number | intake | Neonatal | d1-d7, | | | d1-d24, |
| | of | per litter*, | mortality | kg per | Pre-wean | Numbers | kg per |
| Ingredient | animals | g | up to day 7 | litter* | mortality | weaned | litter* |
| placebo | 49 | 5393 | 10.5% | 15.7 | 12.6 % | 12.9 | 66.5 |
| calcium | 41 | 5565 | 8.4% | 16.9 | 9.6 % | 13.2 | 70.0 |
| gluconate | | | | | | | |

*corrected for litter size.

Colostrum samples were collected just after the first piglet was born and were analyzed for total solids, protein, and lactose, using Brix refractometry. Brix refractometry is a method to measure the concentration of solutes in a solution, based on the refraction of light, and is widely used to analyze milk samples in the dairy industry (for example: Lokke M M, Engelbrecht R, Wiking L. Covariance structures of fat and protein influence the estimation of IgG in bovine colostrum. Journal of Dairy Research (2016), 83: 58-66). This method was validated for sows by comparing Brix values to wet chemistry results in 60 previously collected colostrum samples, which showed a sufficient correlation for total solids (r=0.9), for protein (r=0.98), and for lactose (r=0.7). Total solids is the sum of protein, fat, and lactose. Correlation with fat as such was insufficient to provide reliable data.

The total output of nutrients (protein, lactose, or total solids) per sow was calculated by multiplying the volume of colostrum per sow (Table 2) with the concentration (Table 3) of each nutrient.

Sows supplemented with calcium gluconate had a 6-10% higher output of total solids, total protein and lactose (Table 3), meaning that their litters had access to a greater amount of nutrients.

It as assumed that sows supplemented with gluconate also had higher output of immunoglobulins, since total protein is strongly correlated with colostrum content of IgG (Hasan S M K, Junnikkala S, Valros A, Peltoniemi O, and Oliviero C. Validation of Brix refractometer to estimate colostrum immunoglobulin G content and composition in the sow. Animal (2016), 10: 1728-1733).

TABLE 3

| | | | | Total output | | |
|---|---|---|---|---|---|---|
| | Concentration of solutes in | | | of nutrients per sow¹ | | |
| | colostrum | | | Total | Total | Total |
| | Total | | | solids | protein | lactose |
| | solids², | Protein, | Lactose, | per sow, | per | per sow, |
| Ingredient | % | % | % | g | sow, g | g |
| placebo | 25.4% | 17.9% | 2.3% | 1344 | 948 | 124 |
| calcium | 25.0% | 17.5% | 2.4% | 1437 | 1008 | 136 |
| gluconate | | | | | | |

¹Total output = volume of colostrum × concentration
²Total solids = the total of fat, protein, and lactose

### Example 2

From day 109 of gestation up to parturition (day 115 of gestation), and subsequently through the whole period of lactation from parturition until weaning (25 days after parturition), sows of various parities were fed a top dress of 100 g/day on top of their normal diet. Sows were allocated to either the placebo group (receiving placebo top dress), or a group that received a top dress containing 4 g/100 g top dress calcium gluconate, making sure that sows with different parities were distributed evenly across the two groups. The placebo top dress was formulated from barley, wheat, corn, wheat bran, molasses, vegetable oil, and sugar. In the top dress containing calcium gluconate, 4% (4 g per 100 g) of the placebo top dress was replaced with calcium gluconate (Table 1).

TABLE 4

| | Daily dose of |
|---|---|
| | calcium gluconate |
| Treatment | in 100 g top dress |
| Placebo (n = 37) | 0 g |
| Calcium gluconate (n = 34) | 4 g |

During gestation, sows were fed the normal diet at approximately 3 kg/day. In lactation, all sows were fed the normal diet at an increasing level following a set feeding scheme, starting from 3 kg after parturition, and increasing by 0.5 kg each day to the maximum intake capacity of each sow. The allowance was capped at a maximum of 9 kg/day.

At parturition, piglets were weighed immediately as they were born. One day after parturition, litter size was standardized by cross-fostering, to 14 or 15 piglets per litter. Piglets were only cross-fostered to sows within the same treatment. As a measure of milk production, the weight gain of the litter was measured at 7 days after parturition, and again at 21 days of lactation, just before piglets were weaned (24 days of lactation). Piglets did not receive any additional feed such as creep feed or milk replacer during lactation. When analyzing the data, litter weight gain was corrected for litter size.

Sows gave birth to 14.9 live born piglets on average. Sows receiving calcium gluconate had greater litter weight gain over the whole lactation period (67.1 kg vs 62.1) and weaned more piglets compared to placebo sows (Table 5). Survival of piglets throughout the whole lactation period was greater in litters from sows that were supplemented with calcium gluconate. As a result, litter size at weaning was 0.4 greater in sows supplemented with calcium gluconate top dress compared to those receiving the placebo top dress. Part of the increase in litter gain in sows receiving calcium gluconate was presumably due to the larger number of piglets weaned. When correcting for the number of piglets weaned, sows supplemented with gluconate still had a litter gain that was 2.2 kg greater than placebo sows. In conclusion, gluconate supplementation from day 109 of gestation and in lactation increased milk production, litter gain, and increased survival of piglets to weaning (Table 5).

TABLE 5

| | | Calcium |
|---|---|---|
| | Placebo | gluconate |
| Number of sows | 37 | 34 |
| Litter size after standardization | 15.1 | 15.0 |
| (day 1 of lactation), n | | |
| Litter size at weaning, n | 13.1 | 13.5 |
| Mortality in lactation, n | 1.9 | 1.6 |
| Litter weight gain day 1-day 21, kg | 62.1 | 67.1 |

1. A method of feeding a gestating monogastric animal, comprising administering to the animal a composition comprising gluconic acid or an ester or salt thereof, wherein the amount of gluconic acid or an ester or salt thereof in the composition provides a dosage in a range of grams per day.

2. The method according to claim 1, wherein the composition in the form of a top dress formulation, an animal feed, a premix or supplement or animal drinking water.

3. The method according to claim 1, wherein the gestating monogastric animal is a gestating monogastric mammal.

4. The method according to claim 3, wherein the monogastric mammal is a sow.

5. A method for increasing colostrum production of a monogastric animal and/or for increasing milk production of a monogastric animal, comprising administering to the animal a composition comprising gluconic acid or an ester or salt thereof, wherein the amount of gluconic acid or an ester or salt thereof in the composition provides a dosage in a range of grams per day.

6. The method according to claim 5, wherein the colostral and/or milk protein yield is increased; and/or the total solids yield in colostrum and/or milk is increased; and/or colostral and/or milk lactose yield is increased; and/or immunoglubulins yield in colostrum and/or milk is increased.

7. The method according to claim 5, wherein the composition in the form of a top dress formulation, an animal feed, a premix or supplement or animal drinking water.

8. A method for increasing average daily gain of offspring of a monogastric animal and/or for increasing litter weight gain in offspring of a monogastric animal, comprising administering to the animal a composition comprising gluconic acid or an ester or salt thereof, wherein the amount of gluconic acid or an ester or salt thereof in the composition provides a dosage in a range of 0.02-100 grams per day

9. The method according to claim 8, wherein survival of offspring of the monogastric animal is improved; and/or neonatal mortality of offspring of the monogastric animal is reduced; and/or pre-wean mortality of offspring of the monogastric animal is reduced.

10. The method according to claim 8, wherein the numbers of offspring at weaning of a monogastric animal is increased; and/or the number of weaned offspring is increased.

11. The method according to claim 8, wherein the composition in the form of a top dress formulation, an animal feed, a premix or supplement or animal drinking water.
